(19) United States
(12) Patent Application Publication
(10) Pub. No.: US 2017/0065268 A1
(43) Pub. Date: Mar. 9, 2017

(54) SURGICAL RETRACTOR WITH ASYMMETRIC BLADE

(71) Applicant: Thompson Surgical Instruments, Inc., Traverse City, MI (US)

(72) Inventor: David Sindram, Winston-Salem, NC (US)

(21) Appl. No.: 15/254,672

(22) Filed: Sep. 1, 2016

Related U.S. Application Data
(60) Provisional application No. 62/214,736, filed on Sep. 4, 2015.

(51) Int. Cl. A61B 17/02

(52) U.S. Cl. CPC A61B 17/02; A61B 2017/00407

(57) ABSTRACT

A surgical retraction system may include one or more surgical retractor blades connected to a surgical support shaft or frame. Each retractor blade may extend at an angle from said shaft axis and have a transverse bend that forms an acute angle with the shaft axis. Such transverse bend may result in each retractor blade being asymmetrical with respect to said shaft axis. Each retractor blade may further include a tissue contacting side and an opposing side. The tissue contacting side may include holes or other texturing that hold tissue being retracted.

### BACKGROUND

The invention relates to a surgical retractor blade, and more particularly to a retractor blade for use in chest area surgery.

Retraction apparatus are used during surgical operations to access internal organs and bone structures. Such retraction apparatus include surgical retractor blades. Such surgical retractor blades may be placed into an incision of the patient. The blades may hold the incision open in order for a surgeon to access organs and other biological structures via the incision.

### SUMMARY

Various aspects of this disclosure are directed to retraction apparatus and/or surgical retractor blades of such apparatus. For example and without limitation, various aspects of this disclosure provide asymmetric, surgical retractor blades that may better align with patient's anatomy and/or a desired incision shape, than symmetric, surgical retractor blades.

### BRIEF DESCRIPTION OF SEVERAL VIEWS OF THE DRAWINGS

FIG. 1 shows an incision line generally following a bottom edge of the rib cage of a patient.

FIG. 2 shows an embodiment of a surgical retraction system that may be used to open an incision such as an incision along the incision line of FIG. 1.

FIG. 3 provides a perspective view for a surgical retractor blade of the surgical retraction system of FIG. 2.

FIG. 4 provides another perspective view for a surgical retractor blade of the surgical retraction system of FIG. 2.

FIGS. 5 and 6 provide additional perspective views for a lower blade portion of the surgical retractor blade of FIG. 2.

FIG. 7 provides a perspective view for another surgical retractor blade, which may be a mirror image of the surgical retractor blade of FIGS. 4-6.

FIG. 8 depicts a bendable area of the surgical retractor blades of FIGS. 4-7.

FIG. 9 provides an additional perspective view for the surgical retractor blade of FIGS. 2-6.

FIG. 10 depicts a silhouette-like shape of a cross-sectional side view of the retractor blade of FIGS. 4-6.

FIG. 11 depicts overlapping portions used to form an upper blade portion of the surgical retractor blade of FIG. 2.

### DETAILED DESCRIPTION OF THE INVENTION

The following discussion presents various aspects of the present disclosure by providing examples thereof. Such examples are non-limiting, and thus the scope of various aspects of the present disclosure should not necessarily be limited by any particular characteristics of the provided examples. In the following discussion, the phrases “for example,” “e.g.,” and “exemplary” are non-limiting and are generally synonymous with “by way of example and not limitation,” “for example and not limitation,” and the like.

As utilized herein, “and/or” means any one or more of the items in the list joined by “and/or”. As an example, “x and/or y” means any element of the three-element set {(x), (y), (x, y)}. In other words, “x and/or y” means “one or both of x and y.” As another example, “x, y, and/or z” means any element of the seven-element set {(x), (y), (z), (x, y), (x, z), (y, z), (x, y, z)}. In other words, “x, y and/or z” means “one or more of x, y, and z.”

The terminology used herein is for the purpose of describing particular examples only and is not intended to be limiting of the disclosure. As used herein, the singular forms are intended to include the plural forms as well, unless the context clearly indicates otherwise. It will be further understood that the terms “comprises,” “includes,” “comprising,” “including,” “has,” “have,” “having,” and the like when used in this specification, specify the presence of stated features, integers, steps, operations, elements, and/or components, but do not preclude the presence or addition of one or more other features, integers, steps, operations, elements, components, and/or groups thereof.

It will be understood that, although the terms first, second, etc. may be used herein to describe various elements, these elements should not be limited by these terms. These terms are only used to distinguish one element from another element. Thus, for example, a first element, a first component or a first section discussed below could be termed a second element, a second component or a second section without departing from the teachings of the present disclosure. Similarly, various spatial terms, such as “upper,” “lower,” “side,” and the like, may be used in distinguishing one element from another element in a relative manner. It should be understood, however, that components may be oriented in different manners, for example a component may be turned sideways so that its “top” surface is facing horizontally and its “side” surface is facing vertically, without departing from the teachings of the present disclosure.

In the drawings, various dimensions (e.g., layer thickness, width, etc.) may be exaggerated for illustrative clarity. Additionally, like reference numbers are utilized to refer to like elements through the discussions of various examples.

The discussion will now refer to various example illustrations provided to enhance the understanding of the various aspects of the present disclosure. It should be understood that the scope of this disclosure is not limited by the specific characteristics of the examples provided and discussed herein.

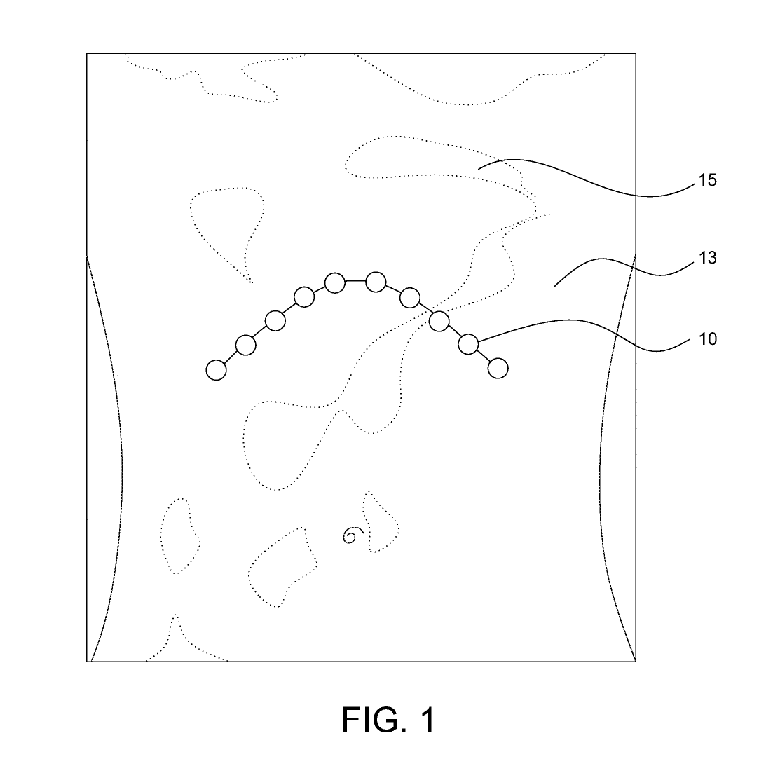

Referring to FIG. 1, a line 10 represents a line along which a surgical incision is to be made into the body of a patient 13. The incision is made to obtain access to the thoracic and/or abdominal cavity during, for example, a liver transplant procedure. The relative location of the patient's liver is generally shown at 15. The incision is curved or arcuate in shape, as shown, bending downwardly on each side of the patient's chest and generally following the costal margin which is the lower edge of the chest (thorax) formed by the bottom edge of the rib cage.

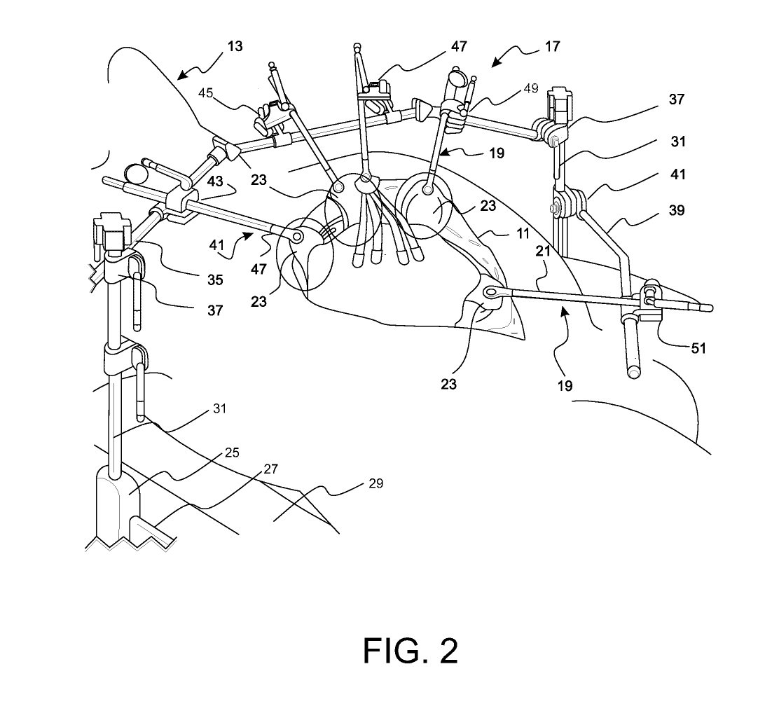

As shown in FIG. 2, a surgical retraction system 17 is used to open the body at incision 11 in order to assist the surgeon in obtaining access to the thoracic and/or abdominal cavity. Retraction system 17 includes a number of retractors 19 each having an arm or handle 21 (with or without a joint attachment 22) and a blade 23. Blade 23 is placed in the open incision and then pulled outwardly to retract the body tissue.

Retraction system 17 may include a pair of rail clamps 25 (one shown), and each rail clamp 25 being secured to one of a pair of horizontal rails 27 located on each side of a conventional operating table 29. A pair of posts 31 each extending vertically from one adjustable rail clamp 25 provides support for a cross bar 35. Cross bar 35 is secured to one of the posts 31 by a universal joint clamp 37. In addition, one or more lateral arms 39 may be secured to post 31 via a universal clamp 41 to provide further supporting structure in the retraction system 17. The cross bar 35 and lateral arm 39 generally define a frame to retractors 19 may be attached.

As shown in FIG. 2, retractor joints 43, 45, 47, 49 and 51 may be disposed along cross bar 35 and along lateral arm 39 for rigidly securing any number of retractors 19 to cross bar 35 or to lateral arm 39.

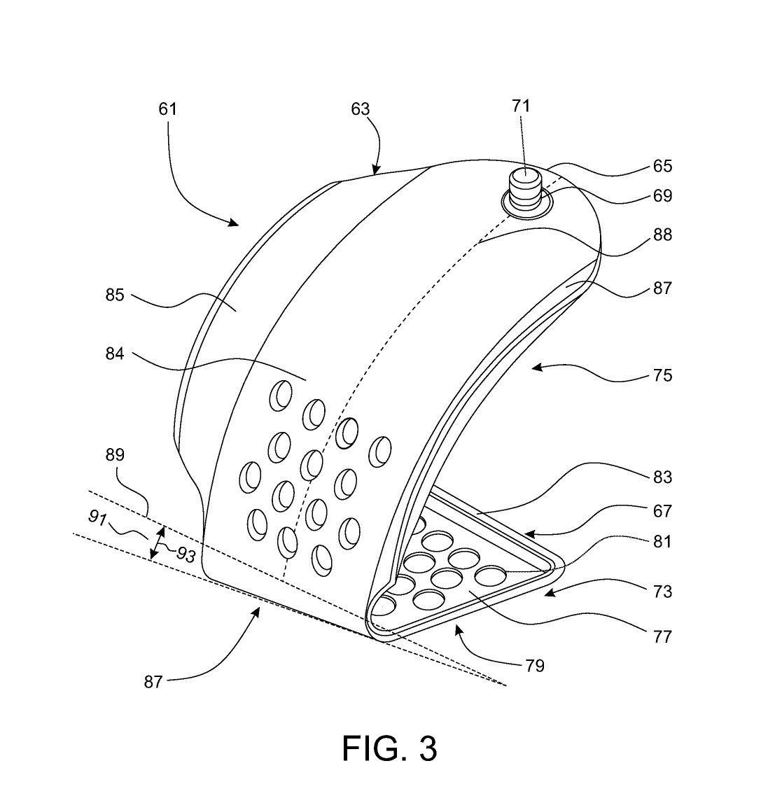

Referring now to FIG. 3, a surgical retractor blade 61 is formed in a particular asymmetric shape for use in retraction system 17. Retractor blade 61 includes a blade body 63 having a proximal end 65 for attachment to retractor arm or handle 21 (FIG. 2) and a distal end 67. Blade body 63, including distal end 67, contacts the anatomy tissue to pull or retract the tissue. Blade body 63 and distal end 67 are to be placed into the surgical incision 11 (FIG. 2) and then retracted outwardly to open the surgical site.

The proximal end 65 of blade body 63 includes a connector 69 such as, for example, a connection nipple for attachment of retractor blade 61 to retractor arm or handle 21. While depicted as a connection nipple, the retraction system 17 may include other types of connectors 69 which connect or attach blade body 63 to retractor arm or handle 21. As shown, the connection nipple of connector 69 may be cylindrical in shape having an axis 71 about which the blade may move, swivel, or be locked, relative to retractor arm or handle 21. In some embodiments, the connector 69 may detachably couple the blade body 63 to the retractor arm 21. In other embodiments, the connector 69 may permanently attach the blade body 63 to the retractor arm 21.

Blade body 63 includes a lower blade portion 73 and an upper blade portion 75. Lower blade portion 73 is planar in shape having a flat top surface 77 and a flat bottom surface 79. Top surface 77 serves to seat against the incision tissue and rib cage anatomy during retraction movement of blade 61. Top surface 77 may be textured by a plurality of holes 81 passing between top surface 77 and bottom surface 79. The textured surface 77 provides a gripping function with respect to the anatomy and tissue of the patient. Other types of textured surface may be used on top surface 77 instead of holes 81, as will suggest itself, and additional holes may be placed in upper blade portion 75.

Lower blade portion 73 may include a lip member 83 which turns upwardly at the distal end 67 of the blade body for extending under the rib cage. Lip member 83 turns upwardly at an angle (for example, 90 degrees or 45 degrees) from the top surface 77 of the lower blade portion, as best seen in FIG. 6. Lip 83 provides a gripping or holding function with respect to the body tissue of the patient.

Referring again to FIG. 3, upper blade portion 75 includes a central area 84 and a pair of side panels 85, 87. Central area 84 is generally curved in shape and integrally connected to lower blade portion 73. Side panels 85, 87 extend outwardly from central area 84 and flare upwardly to provide smooth curved side edges for holding the retracted tissue and evenly distributing the retraction force onto the tissue. As shown, upper blade portion 75 extends along a longitudinal line 88 from a retractor arm connector 69 toward the lower blade portion 73. In some embodiments, the longitudinal line 88 extends along an axis of symmetry of the upper blade portion 75.

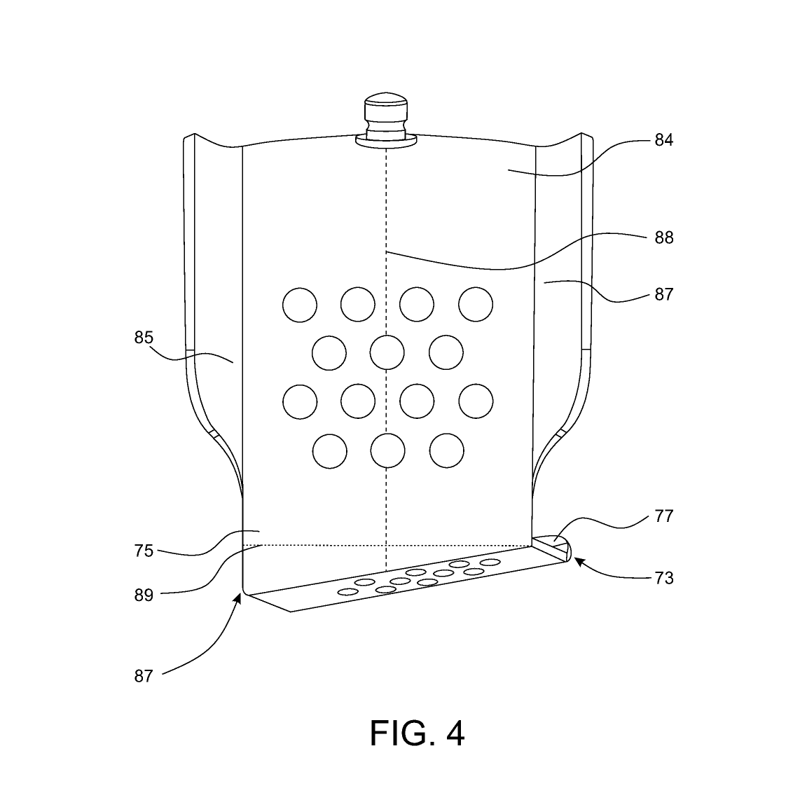

Lower blade portion 73 extends outwardly from upper blade portion 75 in a wide transverse bend 87. Bend 87 provides an asymmetric configuration of the lower blade portion 73 and the upper blade portion 75. Rather than placing the bend 87 relative to a horizontal line 89 perpendicular to axis 88 which would keep the blade body 63 symmetric, bend 87 is placed relative to a line 91 forming an acute angle 93 with horizontal line 89 causing the blade body to be asymmetric. Thus, the lower blade portion 73 is tilted relative to upper blade portion 75 by an angle represented by angle 93. See also FIG. 4 showing the angle of bend 87. The particular angle is provided in relation to the costal margin and may be defined according to the size and shape of the patient.

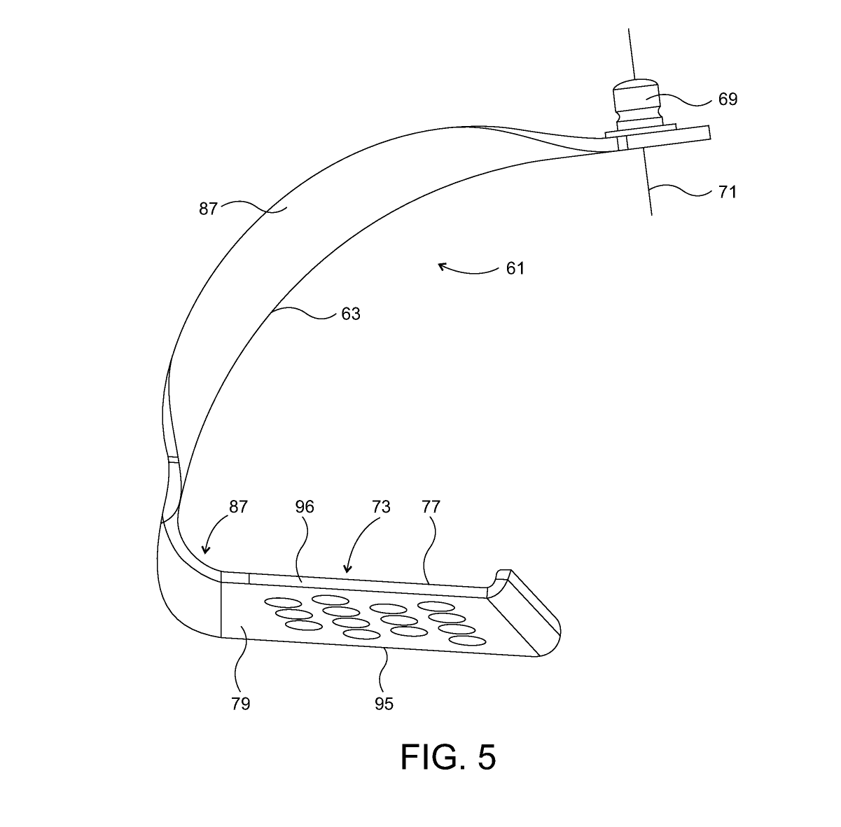

Referring now to FIGS. 5 and 6, lower blade portion 73 is shown tilted with its side 95 (FIG. 5) tilted downwardly relative to its side 96 and revealing its bottom surface 79. Blade body 63 extends outwardly from connection nipple 69 and its axis 71, and then sweeps back toward axis 71 in a wide transverse bend forming bend 87. Bend 87 is formed in a circular arc with bend 87 configured so as to cause asymmetric disposal of lower blade portion 73 relative to the upper blade portion 75. As shown in FIG. 4, the transverse bend 87 is asymmetric in that the plane of top surface 77 is tilted. This downward tilt of lower blade portion 73 allows the top surface 77 to more closely follow the costal margin and curvature of the rib cage, as well as incision 11 of FIG. 2. In addition, the asymmetry aids to prevent blade 61 from slipping out of place.

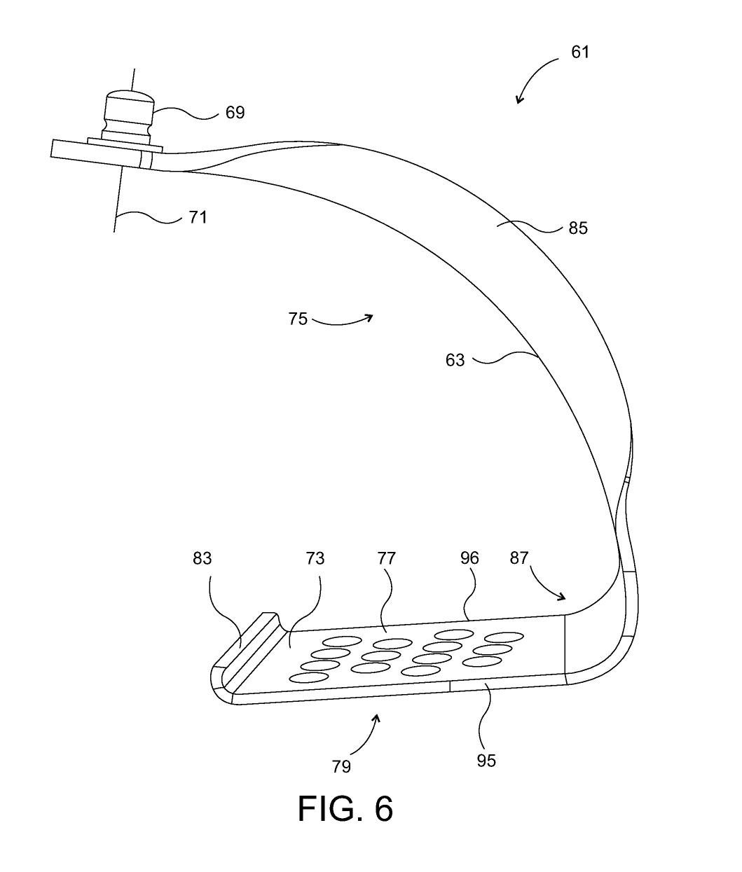

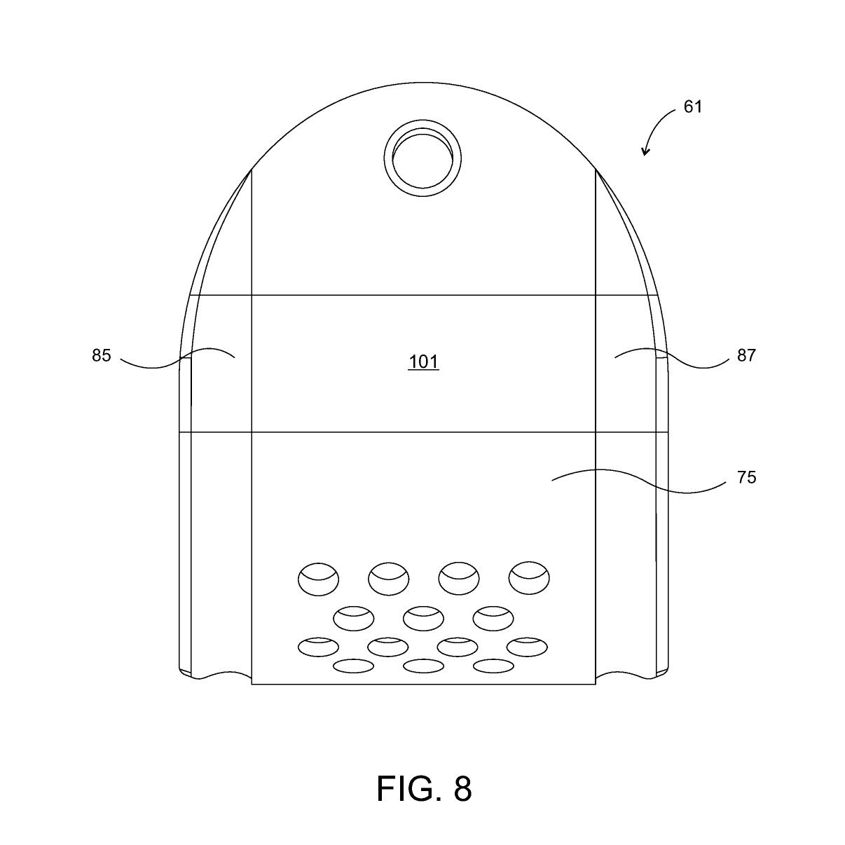

Referring to FIG. 7, a second asymmetric retractor blade 62 is shaped as a mirror image of retractor blade 61 (FIG. 5). As seen by FIG. 5, retractor blade 61 has its side 95 angling downward for positioning on the patient's right side of the rib cage (seen on the left when looking at FIG. 1). The other retractor blade 62 (FIG. 7) has its side 98 angling downward for positioning on the patient's left side of the rib cage (seen as the right when looking at the FIG. 1). As such, a lower portion 73 of retractor blade 62 is offset from it upper portion 75 in a direction that is opposite an offset direction of the lower portion 73 to its upper portion 75 of retractor blade 61. See, e.g., FIG. 9 which depicts the offset of the lower portion 73 of blade 61 with respect to upper portion 75.

Two retractor blades 61, 62 are used in retraction system 17. The rib cage is lifted up away from the body and retracted back (in the direction of the patient's head). The retractor blades 61, 62 hold and retract flesh, skin muscles, the rib cage, etc. to expose and to gain access to the area beneath and around the rib cage. Of course, the retractor blades 61, 62 may be used to perform surgical procedures on other areas of the body.

The asymmetric shape provides less traumatic retraction pressure to the incision, creating a more even or natural alignment of the blade onto the tissue during retraction. This is because the blade is more closely matching to the anatomy.

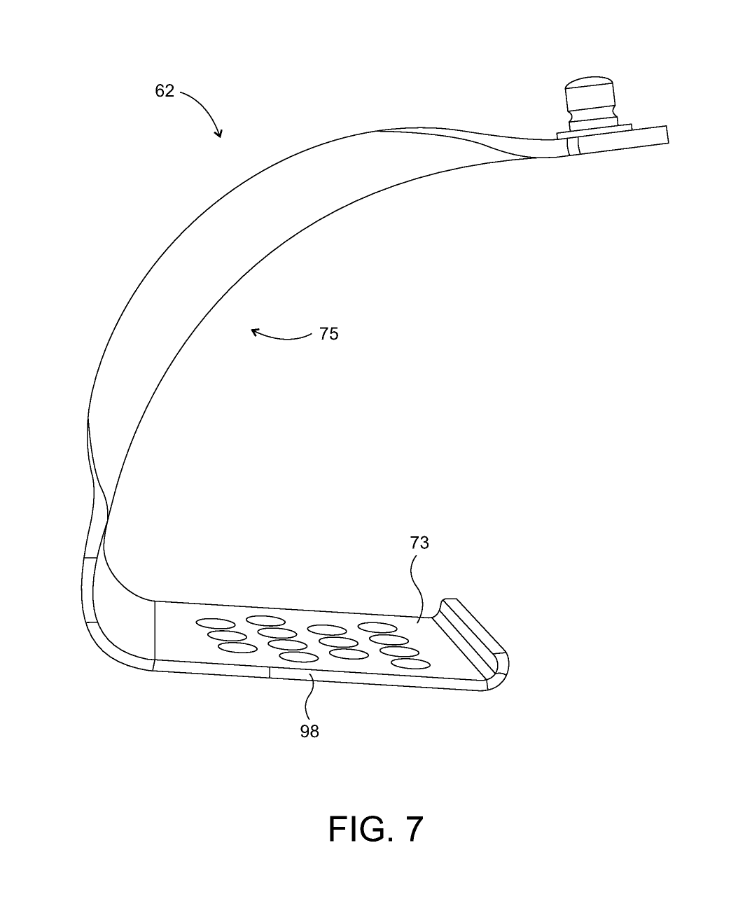

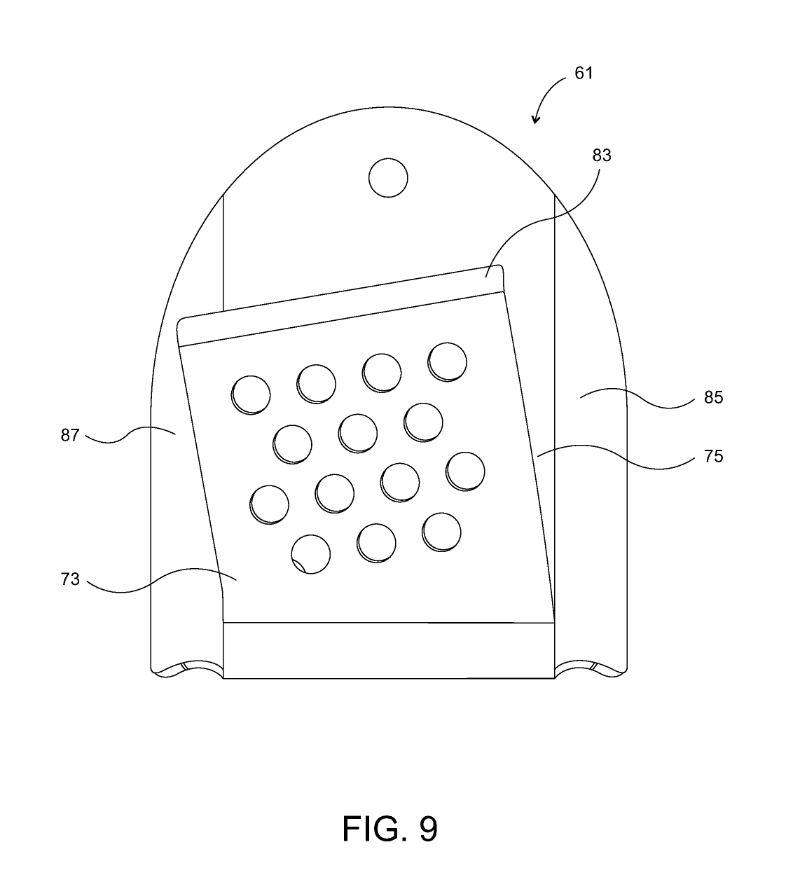

Referring to FIG. 10, a silhouette-like shape of a cross-sectional side view of the retractor blade 61 is shown. The upper portion 75 of blade 61 may include an area 101 that is formed of a malleable material, for example, a malleable metal. Area 101 is shown in FIG. 8. This area 101 is bendable out of its original shape and into a formed shape without breaking or cracking of the blade 61. Such an area 101 allows the surgeon to form the amount of curvature and position of the blade during the retraction procedure.

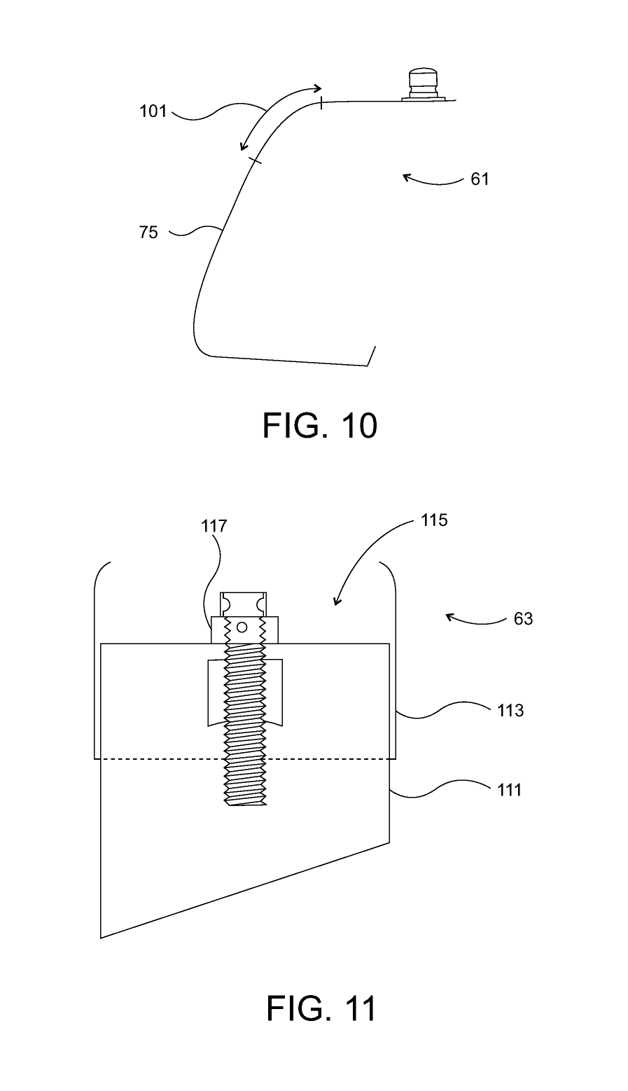

Referring to FIG. 11, the upper portion 75 of blade body 63 may be formed by two overlapping portions 111, 113. The two portions 111, 113 are movable relative to one another to adjust size/depth, and assist with gripping of the tissue to prevent blade slip, via for example, a ratchet mechanism 115. A rotatable knob 117 is rotatable by the user to ratchet to adjust the relative position of the two overlapping portions 111, 113. This allows multiple thicknesses and multiple dimensions to the retractor blade. This may be found favorable when used with larger obese patients.

While the foregoing has been described with reference to certain aspects and examples, it will be understood by those skilled in the art that various changes may be made and equivalents may be substituted without departing from the scope of the disclosure. In addition, many modifications may be made to adapt a particular situation or material to the teachings of the disclosure without departing from its scope. Therefore, it is intended that the disclosure not be limited to the particular example(s) disclosed, but that the disclosure will include all examples falling within the scope of the appended claims.

What is claimed is:

1. A surgical retractor blade assembly, comprising:
a retractor arm configured to be attached to a frame of a surgical retraction system; and
an asymmetric retractor blade extending along a longitudinal line from a proximal end coupled to the retractor arm toward a lower portion of the retractor blade, said retractor blade having a transverse bend forming an acute angle with a horizontal line perpendicular to the longitudinal line.

2. The surgical retractor blade assembly of claim 1, wherein the proximal end of the retractor blade comprises a connector configured to connect the retractor blade to the retractor arm.

3. The surgical retractor blade assembly of claim 1, wherein said retractor blade is permanently attached to the retractor arm.

4. The surgical retractor blade assembly of claim 1, wherein said surgical retractor blade assembly swivels relative to a handle.

5. The surgical retractor blade assembly of claim 1, wherein the retractor blade comprises an upper blade portion associated with the proximal end of the retractor blade and a lower blade portion associated with a distal end of the retractor blade, said upper and lower blade portions meeting in at the transverse bend.

6. The surgical retractor blade assembly of claim 5, wherein:
the upper blade portion comprises a central area and a pair of side panels;
the central area is integrally connected to the lower blade portion along the transverse bend;
the pair of side panels extend outwardly from the central area and flare upwardly to provide curved side edges that hold retracted tissue and distribute a retraction force onto the retracted tissue.

7. The surgical retractor blade assembly of claim 5, wherein the upper blade portion includes an area formed of a malleable material that permits bending the upper blade portion, during the retraction procedure, to conform to a patient's anatomy.

8. The surgical retractor blade assembly of claim 5, wherein the longitudinal line lies along an axis of symmetry of the upper blade portion.

9. The surgical retractor blade assembly of claim 5, wherein:
the upper blade portion comprises a first portion, a second portion, and a ratchet;
the first portion and the second portion overlap; and
the ratchet is configured to adjust a size of the retractor blade by moving the first portion relative to the second portion.

10. An asymmetric, surgical retractor blade for retracting tissue of a patient, the retractor blade comprising:
an upper portion; and
a lower portion coupled to the upper portion along a transverse bend;
wherein the upper portion extends along a longitudinal line from a proximal end of the retractor blade toward the lower portion of the retractor blade; and
wherein the transverse bend forms an acute angle with a horizontal line that is perpendicular to the longitudinal line.

11. The surgical retractor blade of claim 10, wherein:
the upper blade portion comprises a central area and a pair of side panels;
the central area is integrally connected to the lower blade portion along the transverse bend; and
the pair of side panels extend outwardly from the central area and flare upwardly to provide curved side edges that hold retracted tissue and distribute a retraction force onto the retracted tissue.

12. The surgical retractor blade of claim 10, wherein the upper blade portion includes an area formed of a malleable material that permits bending the upper blade portion, during the retraction procedure, to conform to a patient's anatomy.

13. The surgical retractor blade of claim 10, wherein the longitudinal line lies along an axis of symmetry of the upper blade portion.

14. The surgical retractor blade of claim 10, wherein:
the upper blade portion comprises a first portion, a second portion, and a ratchet;
the first portion and the second portion overlap; and
the ratchet is configured to adjust a size of the retractor blade by moving the first portion relative to the second portion.

15. A retraction system for retracting tissue of a patient, the retractor system comprising:
a frame;
a first retractor coupled to the frame, the first retractor comprising a first asymmetric retractor blade including a first upper blade portion coupled to a first lower blade portion via a first transverse bend such that the first lower blade portion is offset from the first upper blade portion in a first direction; and
a second retractor coupled to the frame, the second retractor comprising second asymmetric retractor blade including a second upper blade portion coupled to a second lower blade portion via a second transverse bend such that the second lower blade portion is offset from the first upper blade portion in a second direction opposite the first direction.

16. The retraction system of claim 15, wherein the first retractor blade is a mirror image of the second retractor blade.

17. The retraction system of claim 15, wherein:
the first upper blade portion comprises a first central area and a pair of first side panels;
the first central area is integrally connected to the first lower blade portion along the first transverse bend; and
the pair of first side panels extend outwardly from the first central area and flare upwardly to provide curved side edges that hold retracted tissue and distribute a retraction force onto the retracted tissue.

18. The retraction system of claim 15, wherein the first upper blade portion includes an area formed of a malleable material that permits bending the first upper blade portion, during the retraction procedure, to conform to a patient's anatomy.

19. The retraction system of claim 15, wherein:
the first upper blade portion comprises a first portion, a second portion, and a ratchet;
the first portion and the second portion overlap; and
the ratchet is configured to adjust a size of the first retractor blade by moving the first portion relative to the second portion.
